(19) United States
(12) Patent Application Publication
(10) Pub. No.: US 2005/0069958 A1
(43) Pub. Date: Mar. 31, 2005

(54) Method for simultaneous evaluation of a sample containing a cellular target and a soluble analyte

(76) Inventors: Rhonda A. Mills, Pembroke Pines, FL (US); Jorge A. Quintana, Tavernier, FL (US); John A. Maples, Miami, FL (US); Paul M. Scibelli, Miami, FL (US)

Correspondence Address: BECKMAN COULTER, INC., P.O. BOX 169015, MAIL CODE 32-A02, MIAMI, FL, 33116-9015, US

(21) Appl. No.: 10/672,477

(22) Filed: Sep. 26, 2003

(51) Int. Cl. G01N 33/53; G01N 33/567

(52) U.S. Cl. 435/7.2

(57) ABSTRACT

A method for evaluating a biological sample containing at least one cell type bearing at least one cellular target and at least one soluble analyte, involves adding to a single container the sample with (i) at least one soluble ligand that binds the cellular target, (ii) either at least one soluble ligand that binds the soluble analyte or at least one competing soluble analyte; and (iii) a solid phase capture medium that binds directly to the soluble analyte, indirectly to the soluble analyte, or to the soluble ligand that binds to the soluble analyte. Various complexes involving the capture medium and soluble analyte, the capture medium and the soluble ligand, and/or the cellular target and a soluble ligand are simultaneously analyzed without physically separating the complexes.

### BACKGROUND OF THE INVENTION

The present invention related to assay methods for quantitative and qualitative evaluation of biological samples containing both cellular targets or analytes and soluble targets or analytes.

The ability to detect and/or measure a wide variety of targets, analytes, molecules, chemical compounds and complexes and the like in a variety of biological samples or products has significant use the diagnosis of disease, the treatment of disease, the monitoring of the efficacy of therapy, research in molecular biology, the detection and monitoring of water purity, product contamination, and other fields. A number of different types of assays have evolved depending upon the identity and state of the target to be analyzed, e.g., a target immobilized on or in a substrate (such as a cell) or a soluble target, a target that is proteinaceous, or a chemical compound, etc. Thus, immunoassays exist that identify proteinaceous targets using antibodies; competition immunoassays identify targets by allowing the target to compete for binding to a limited amount of an antibody with a known amount of a labeled antigen. The amount of labeled antigen bound to the antibody is inversely proportional to the amount of antigen in the sample. Immunometric assays employ a labeled antibody and the amount of labeled antibody associated with the target is measured as directly proportional to the amount of target available in the sample. Cytometric assays identify targets by size, shape, charge, light diffraction or reflection, or other means.

Generally, assays to detect targets by immobilizing a ligand on a solid support and forming a complex between the target and the bound ligand require different processing steps than assays employing soluble ligands to detect. Further, in assays performed on blood samples, phagocytosis of the solid support particles by myeloid cells in the sample can introduce error into the assays. Most known assays also involve multiple processing steps, e.g., lysing, washing, and physically separating components formed in the sample prior to detecting the appropriate ligand or label. Further known assays generally employ a wash step to remove bound analyte from soluble analyte and are performed in a serum, plasma or media matrix devoid of cells. Additional steps of washing, lysing and separating introduce inaccuracies by reducing the amount of target inadvertently. The need to perform a number of different assays on a single rare or small sample is often another problem experienced with assays, which leads to depletion of the sample material and an increase in the costs of the assays themselves.

Various assays are described in the following references: Lindmo T, et al, J. Immunol. Meth., 1990 126:183-189; Frengen J et al, J. Immunol. Meth., 1995 178:131-140; Frengen J et al, J. Immunol. Meth., 1995 178:141-151; U.S. Pat. Nos. 4,572,0284,376,110; 5,006,459; 5,168,044; 5,426,029; 5,525,461; 5,567,627; 5,756,011; 5,811,525; 5,981,180; 6,268,155; and U.S. patent publication Ser. No. 2002/0076833.

There remains a need in the art for more efficient methods of analyzing multiple analytes in a single sample, which methods can reduce the steps performed on the samples, thereby improving accuracy and cost effectiveness, and preserving rare samples.

### SUMMARY OF THE INVENTION

In one aspect, the invention provides a method enabling the evaluation of a biological sample containing at least one cell type bearing at least one cellular target and at least one soluble analyte. The steps of the method involve adding to a single container the sample with (i) at least one soluble ligand that binds the cellular target, (ii) at least one soluble ligand that binds the soluble analyte or at least one competing soluble analyte, preferably labeled with a detectable marker; and (iii) a solid phase capture medium that binds directly to the soluble analyte, indirectly to the soluble analyte, or to the soluble ligand that binds to the soluble analyte. Various complexes form between and among the soluble ligands employed in the assay method, the soluble analytes (competing or naturally occurring), the cellular target or analyte and the capture medium described above. The complexes include a complex that forms between the cellular target and at least one soluble ligand. Another complex forms between the capture medium bound directly to the soluble analyte (competing or naturally occurring in the sample). Still another complex may form between the capture medium bound indirectly to the soluble analyte. Another complex may form between the capture medium bound to the soluble ligand that is bound to the soluble analyte. Thereafter, the sample with its variety of complexes is analyzed without physically separating the complexes. The method steps may depend upon the type of assay method employed, e.g., sandwich assay, competitive assay, or immune-complex assay.

In another aspect, the invention provides a method for diagnosing sepsis or monitoring the progress thereof by performing the above-described novel simultaneous evaluation method.

In still another aspect, the invention provides a method for diagnosing autoimmune disease or monitoring the progress thereof by performing above-described novel simultaneous evaluation method.

In yet a further aspect, the invention provides a method for diagnosing cardiovascular disease or monitoring the progress thereof by performing the above-described novel simultaneous evaluation method.

Another aspect of this invention is a method for differential diagnosis of viral and bacterial infections or monitoring the progress thereof by performing the above-described novel simultaneous evaluation method.

In another aspect of this invention, a method for evaluating the toxicity or safety of water or other solutions involves performing the above-described novel simultaneous evaluation method.

Yet a further embodiment of the present invention involves a kit for performing the various methods described above that comprises at least one soluble ligand that binds a cellular target in the sample. The kit also provides either at least one soluble ligand that binds a soluble analyte in the sample or at least one competing soluble analyte that is preferably labeled. The kit also provides a solid phase capture medium that binds directly to the soluble analyte, indirectly to the soluble analyte, or to the soluble ligand that binds to the soluble analyte.

Other aspects and advantages of the present invention are described further in the following detailed description of the preferred embodiments thereof.

### BRIEF DESCRIPTION OF THE DRAWINGS

FIG. 1 is a schematic diagram depicting the method of the present invention employing “sandwich assay” steps, in which the capture medium is coated with a ligand for the soluble target which is an antibody, a second soluble ligand (antibody) that binds to the cellular target and which is associated with a fluorescent label FL1, and a third soluble ligand (antibody) that binds the soluble target and is associated with a second fluorescent label FL2. The simultaneous evaluation of the complexes formed is demonstrated by a flow cytometry graph of forward light scatter vs. log of side scatter and two graphs of the cpm of the two fluorescent labels.

FIG. 2A is a schematic diagram depicting the method of the present invention employing “competitive inhibition assay” steps. The capture medium is coated with soluble analyte. Also employed in the method are a soluble ligand for the cellular target that is an antibody and which is associated with a fluorescent label FL1, and a second soluble ligand (antibody) that binds to the analyte in the sample or on the bead, and which is associated with a fluorescent label FL2. The potential complexes formed are (1) a complex formed by the soluble ligand-FL1 and the cellular target, (2) a complex formed by the capture medium with immobilized soluble analyte and the second soluble ligand-FL2 (that has not bound to soluble analyte in the sample), and (3) the soluble analyte in the sample, if any, and the second soluble ligand-FL2. The simultaneous evaluation of the complexes (1) and (2) formed is demonstrated by a flow cytometry graph of forward light scatter vs. log of side scatter and two graphs of the cpm of the two fluorescent labels. The measurement of the FL2 on the capture medium-immobilized analyte-ligand-FL2 complex is inversely proportional to the amount of soluble analyte in the sample due to competition for binding between the immobilized analyte and the soluble analyte in the sample.

FIG. 2B is schematic diagram depicting the method of the present invention employing alternative “competitive inhibition assay” steps. In this alternative, the capture medium is coated with the soluble ligand that binds the soluble analyte. Also employed in this method is a soluble ligand for the cellular target that is an antibody and which is associated with a fluorescent label FL1. Another component of the method is a soluble analyte that is associated with a fluorescent label FL2. The potential complexes formed are (1) a complex formed by the soluble ligand-FL1 and the cellular target, (2) a complex formed by the capture medium with immobilized ligand for the soluble analyte and the soluble analyte-FL2, and (3) a complex formed by the capture medium with immobilized ligand for the soluble analyte and the soluble analyte in the sample, if any. The simultaneous evaluation of the complexes (1) and (2) formed is demonstrated by a flow cytometry graph of forward light scatter vs. log of side scatter and two graphs of the cpm of the two fluorescent labels. The measurement of the FL2 on the capture medium-immobilized ligand-bound analyte-FL2 complex is inversely proportional to the amount of unlabeled soluble analyte in the sample.

FIG. 3 is a schematic diagram depicting the method of the present invention employing “immune complex” steps. The bead is coated with streptavidin. A first ligand (antibody) to the cellular target is associated with a fluorescent label FL1. A second ligand (antibody) to the cellular target is associated with a fluorescent label FL2. A third ligand (antibody associated with biotin) is targeted to the soluble analyte. The simultaneous evaluation of the complexes formed is demonstrated by a flow cytometry graph of forward light scatter vs. log of side scatter and two graphs, one showing the cpm of fluorescent label FL1, and the other showing the cpm of fluorescent label FL2.

### DETAILED DESCRIPTION OF THE INVENTION

The method of the present invention answers the need in the art by providing for the simultaneous evaluation (detection and/or measurement) of both soluble and bound targets in a sample. The method involves generally the analysis of a sample, which contains at least one target bound to a larger structure and at least one soluble analyte, which is unbound and free in the solution of the sample.

The general steps of the method involve adding to a single container the sample with (i) at least one soluble ligand that binds the cellular target, (ii) at least one soluble ligand that binds the soluble analyte or at least one competing soluble analyte that is preferably associated with a detectable label; and (iii) a solid phase capture medium that binds directly or indirectly to the soluble analyte or to the soluble ligand that binds the soluble analyte. After appropriately incubating the sample with these additives, the sample is simultaneously analyzed without physically separating the different complexes that form within the sample. For example, one potential complex forms between the cellular target and at least one soluble ligand. Another potential complex forms between the capture medium bound directly to the soluble analyte (either labeled or unlabeled). Generally this direct binding involves a capture medium having immobilized thereon at least one ligand (e.g., a monoclonal antibody) that binds to the analyte. Still another complex may form between the capture medium bound indirectly to the soluble analyte. In this instance, the capture medium has coated thereon a ligand (e.g., biotin) that binds to another ligand (e.g., streptavidin) that is attached to a ligand for the soluble analyte (e.g., a monoclonal antibody that binds the analyte). Another complex may form between the capture medium bound to the soluble ligand that is bound to the soluble analyte. In this instance, the capture medium has coated thereon the soluble analyte, which binds the soluble ligand for the analyte.

It should be understood that one or more of the ligands employed in these methods are labeled with one or more detectable markers, as described in more detail below. In certain competitive inhibition assay formats, one or more soluble analytes employed in these methods are labeled with one or more detectable markers, as described in detail below. The lack of a physical separation step, i.e., the ability to measure the relevant complexes in the same container, in this method provides a valuable advantage in terms of efficiency and time in obtaining results of analysis, and further provides an advantage of preserving a small or rare sample, by using as little sample as possible.

A. The Sample

Preferably the sample is a biological sample, in which the bound target is a cell bearing at least one cellular target, and having at least one soluble analyte. The biological sample preferably contains cells of various types of biological tissue. For example, certain biological samples include, without limitation, whole blood, saliva, urine, synovial fluid, bone marrow, cerebrospinal fluid, vaginal mucus, cervical mucus, sputum, semen, amniotic fluid, cell lines, cell-containing exudates, cell-containing media, cell-containing buffer, bacterial samples, viral sample, and other exudates from a patient containing bacteria or virus. Such samples may further be diluted with saline, buffer or a physiologically acceptable diluent. Preferably such dilution occurs before addition of the soluble ligand(s) or of the competing soluble analyte(s).

In such biological sample, the cell type bearing the cellular target may be biological cells, particularly mammalian hematological or blood cells, and also all vertebrate or invertebrate cells, insect cells, bacterial cells, parasites, yeast or fungal cells, algal or other plant cells, etc. Also included in this definition are viruses, virus-like particles, parasites, and essentially any biological colloidal particle that has on its surface a receptor or antigen (i.e., an analyte) for which there exists a counter-receptor ligand or specific binding partner. The present invention is described specifically below using mammalian blood cells, specifically one or more of red blood cells and white blood cells. Among the white blood cells that may be present include, without limitation, granulocytes, macrophages, platelets, lymphocytes, lymphoblasts, blast cells, leukocytes, neutrophils and dendritic cells. Other cell types of use in these methods include, without limitation, fibroblasts, epithelial cells, epidermal cells, embryonic cells, hepatocytes, histiocytes, peritoneal cells, kidney cells, lung cells, sperm cells, oocytes, and normal and cancer cells of other mammalian tissue. The cellular target is generally, a cell surface antigen, an intercellular antigen, nuclear antigen, a fragment thereof, or a mixture of two or more of the preceding targets.

The soluble analyte, which is naturally occurring in such biological sample, or which is alternatively a competing soluble analyte employed as a component of certain embodiments of the methods of this invention, is likely to include, without limitation, a serum marker, a pharmaceutical drug, a protein, a virus, a hormone, a lipid, a nucleic acid sequence, a carbohydrate, a toxin, or an antigen shed from a cell type identified above, or produced or secreted by a mammalian cell, a bacterial cell, a virus, a cell infected by a virus, a cancer cell, a fungus, etc., or a fragment thereof, or a mixture of two or more of the preceding analytes.

Such naturally occurring targets and/or soluble analytes are desirable to detect or quantify due to their relationship to disease states. Thus detection of such targets is useful in diagnosis of disease, or monitoring of therapy, among others.

Still other types of sample which can be evaluated according to the method of this invention include water from any source, manufactured liquids such as gasoline, alcohol, pharmaceutical medicines, perfumes, food products, and the like. The targets and analytes in these samples may include adulterating compounds, such as drugs, poisons, toxins, microbial proteins and the like. Thus such targets are desirably detected as a means of quality control for detecting unwanted contamination or adulteration.

In certain embodiments, the sample can contain additional reagents. For example, where the sample is whole blood, the sample can contain an anti-coagulant, such as those described below. In another embodiment, the sample containing myeloid cells can contain an inhibitor of phagocytosis, such as discussed below. In still another embodiment, the sample can be treated with one or more of a fixative, a phosphatase inhibitor, or a calcium inhibitor.

B. Ligands Useful in the Invention

Generally, the components of the method include ligands that bind either the cellular target or the soluble analyte. By “ligand” is meant a moiety or binding partner that specifically binds to the target on the cell or to the soluble analyte. Such ligands are individually and independently an antibody that binds a cellular antigen, an antibody that binds a soluble antigen, an antigen that binds an antibody already bound to the cellular or soluble antigen; or fragments of such antibodies and antigens that are capable of binding; a nucleic acid sequence sufficiently complementary to a target nucleic acid sequence of the cellular target or soluble analyte to bind the target or analyte sequence, a nucleic acid sequence sufficiently complementary to a ligand nucleic acid sequence already bound to the cellular target or soluble analyte, or a chemical or proteinaceous compound, such as biotin or avidin.

The ligands can be soluble or can be immobilized on the capture medium (i.e., synthetically covalently linked to a bead), as indicated by the assay format. As defined herein, ligands include various agents that detect and react with one or more specific cellular targets or soluble analytes. Examples of ligands within the meaning of the present invention and their analytes include, without limitation, those listed in Table 1.

TABLE 1

| LIGAND | RECEPTOR |
|---|---|
| Antibody | Antigen |
| Natural ligands: cytokine | CK receptor |
| or chemokine | |
| Hormone | Hormone receptor |
| growth factor | Growth factor receptor |
| Secondary reagents: | Biotinylated antibody - antigen |
| streptavidin | |
| Antibody | Antibody - antigen |
| Synthetic peptides | Receptor |
| Solubilized natural ligands: | Receptor |
| Counter- receptor | |
| CTLA-4 | B7 (CD80/86) |
| Lectins (agglutinins) | Complementary carbohydrate or |
| | oligosaccharide on cell-surface |
| | Glycoprotein |
| MHC-peptide complex | T cell receptor (TCR) |
| Oligonucleotide | Complementary sequences in |
| | nucleic acids, DNA or RNA |

Methods useful for construction of such ligands are known to those of skill in the art. All such ligands are characterized by the desired ability to bind the specified target or analyte, whether it is soluble or bound to a cell. In one preferred embodiment, the ligand of the invention is a component that preferentially binds to all or a portion of a cell surface receptor. Thus, a ligand useful in this embodiment of the invention may be an antibody or a functional fragment thereof capable of binding to a cell surface receptor on a WBC population. Such antibodies or fragments include polyclonal antibodies from any native source, and native or recombinant monoclonal antibodies of classes IgG, IgM, IgA, IgD, and IgE, hybrid derivatives, and fragments of antibodies including Fab, Fab′ and F(ab′)2, humanized or human antibodies, recombinant or synthetic constructs containing the complementarity determining regions of an antibody, an Fc antibody fragment thereof, a single chain Fv antibody fragment, a synthetic antibody or chimeric antibody construct which shares sufficient CDRs to retain functionally equivalent binding characteristics of an antibody that binds a desired cell surface receptor, and a binding fragment produced by phage display.

Antibodies used in the examples of this invention were generally obtained by conventional hybridoma methods and purified from ascites fluid by ammonium sulfate (45%) precipitation, centrifugation and affinity chromatography using protein A. The standard process of making monoclonal antibodies is described in G. Kohler and C. Milstein, 1975 Nature, 256: 495-497. Of course, the particular method of making and the type of monoclonal antibody is not limited to such techniques and it is envisioned that any technique for making such antibodies is within the practice of the invention. Any ligand which can bind cellular targets or soluble analytes may be used, since the amplification of fluorescent intensities does not depend on the density of the particular receptor sites on a cell.

Other typical ligands can include, without limitation, a lectin, a hormone, a growth factor, or a synthetic peptide or chemical compound, or portions thereof that can bind the target or analyte. The selection of the ligand is not a limiting factor in this invention. Exemplary ligands are illustrated in the specific embodiments of methods described below and in the examples.

C. Detectable Labels or Markers

Where indicated, the ligands and/or the competing soluble analytes and/or the capture medium employed in the methods of this invention are associated with detectable labels or detectable markers. Detectable labels for attachment to components useful in this invention may be easily selected from among numerous compositions known and readily available to one skilled in the art of diagnostic assays. The reagents, ligands, competing analytes, or capture medium of this invention are not limited by the particular detectable label or label system employed. In some cases, the detectable “label” can include the refractive index of a cell surface or bead.

As used herein, the terms “label” or “marker” generally refers to a molecule, preferably proteinaceous, but also a small chemical molecule that is capable, acting alone, or in concert with other molecules or proteins, of providing a signal, that is detectable either directly or indirectly. In this invention, the marker is associated with the various ligands or competing analytes used in the assays. For example, a detectable label or marker can be a fluorescent label, a luminescent label, a radiolabel, or a chemiluminescent label.

In one embodiment, preferred markers enable detection by emitting a detectable signal of a particular wavelength upon excitation by a laser. Phycobiliproteins, tandem dyes, certain fluorescent proteins, small chemical molecules, and certain molecules detectable by other means can all be considered markers for flow cytometry analyses. See, e.g., the markers listed in Handbook of Fluorescent Probes and Research Chemicals, 6th Ed., R. P. Haugland, Molecular Probes, Inc., Eugene, Oreg. (1996). “Phycobiliproteins” are a family of macromolecules found in red algae and blue-green algae. The biliproteins (the term “biliproteins” is equivalent to the term “phycobiliprotein”) have a molecular weight of at least about 30,000 daltons, more usually at least about 40,000 daltons, and may be as high as 60,000 or more daltons usually not exceeding about 300,000 daltons. The biliproteins will normally be comprised of from 2 to 3 different subunits, where the subunits may range from about 10,000 to about 60,000 molecular weight. The biliproteins are normally employed as obtained in their natural form from a wide variety of algae and cyanobacteria.

The presence of the protein in the biliproteins provides a wide range of functional groups for conjugation to proteinaceous and non-proteinaceous molecules. Functional groups that are present include amino, thiol, and carboxyl. In some instances, it may be desirable to introduce functional groups, particularly thiol groups when the biliprotein is to be conjugated to another protein. Each phycobiliprotein molecule contains a large number of chromophores. An exemplary ligand, e.g., an antibody molecule directly labeled with fluorescein will have between 1 and 3 chromophores associated with it. An antibody molecule (for example) directly labeled by conjugation with a phycobiliprotein may have as many as 34 associated chromophores, each with an absorbance and quantum yield roughly comparable to those of fluorescein.

Examples of phycobiliproteins useful in the present invention are phycocyanin, allophycocyanin (APC), allophycocyanin B, phycoerythrin (PE) and preferably R-phycoerythrin. PE is among the brightest fluorescent dyes currently available. Conjugated to an antibody, PE has been used to detect interleukin-4 in a fluorescent plate assay and found to be the only tested fluorophore that produced adequate signal (M. C. Custer and M. T. Lotze, 1990 J. Immunol. Methods, 128, 109-117).

The tandem dyes are non-naturally occurring molecules that may be formed of a phycobiliprotein and another dye. See, for example, U.S. Pat. No. 4,542,104 and U.S. Pat. No. 5,272,257. Examples of tandem dyes useful in the present invention are phycoerythrocyanin or PC5 (PE-Cy5, phycoerythrin-cyanin 5.1; excitation, 486-580 nm, emission, 660-680 nm) [A. S. Waggoner et al, 1993 Ann. N. Y Acad. Sci., 677:185-193 and U.S. Pat. No. 5,171,846] and ECD (phycoerythrin-texas red; excitation, 486-575 nm, emission, 610-635nm) [U.S. Pat. No. 4,542,104 and U.S. Pat. No. 5,272,257. Other known tandem dyes are PE-Cy7, APC-Cy5, and APC-Cy7 [M. Roederer et al, 1996 Cytometry, 24:191 - 197]. Tandem dyes, PC5 and ECD, have been successfully directly conjugated to monoclonal antibodies by several methods that involve iminothiolane activation of the dye.

Preferably, the ligands and/or competing analytes and/or capture medium of this invention are associated with, or conjugated to fluorescent detectable fluorochromes, e.g., fluorescein isothiocyanate (FITC), phycoerythrin (PE), allophycocyanin (APC), or tandem dyes, PE-cyanin-5 (PC5), PE-cyanin-7 (PC7), and PE-Texas Red (ECD). The biliproteins and tandem dyes are commercially available from various sources including Beckman Coulter, Inc., Miami, Fla., Molecular Probes, Inc., Eugene, Oreg. and Prozyme, Inc., San Leandro, Calif. All of these fluorescent dyes are commercially available, and their uses known to the art.

Still other markers that may be directly conjugated to the components of the methods of this invention and used with the phycobiliproteins or tandem dyes in this invention to add additional numbers of markers (labeled ligands) to the method include small molecules that upon excitation emit wavelengths of less than 550 nm. Such molecules do not overlap with the emissions of the phycobiliproteins. One example of such a marker is fluorescein isothiocyanate (FITC). Others are listed in the Handbook cited above.

Still other markers that may be employed in this method to provide additional colors are the proteins known as the green fluorescent proteins and blue fluorescent proteins; also useful may be markers that emit upon excitation by ultraviolet light.

A marker can be an enzyme that interacts with a substrate to produce the detectable signal. Another marker embodiment can be a protein that is detectable by antibody binding or by binding to a suitably labeled ligand. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to form conjugates with the inhibitor sequences or ligands and provide a visual signal indicative of the presence of the resulting complex in applicable assays. Still other label systems that may be used include nanoparticles or quantum dots.

In another embodiment such markers may preferably be reporter genes that upon expression produce detectable gene products. Such reporter sequences include without limitation, DNA sequences encoding a lux gene, beta-lactamase, a galactosidase enzyme, e.g., beta-galactosidase (LacZ), alkaline phosphatase, thymidine kinase, green fluorescent protein (GFP), chloramphenicol acetyltransferase (CAT), a luciferase enzyme, or a gluconase enzyme.

Still other suitable marker that may be attached to the components of the methods of this invention include membrane bound proteins including, for example, CD2, CD4, CD8, the influenza hemagglutinin protein, a biotin molecule, an avidin molecule, and others well known in the art, to which high affinity antibodies directed thereto exist or can be produced by conventional means. Another class of markers includes fusion proteins comprising a membrane bound protein appropriately fused to an antigen tag domain from, among others, hemagglutinin or a Myc gene. Still other detectable labels may include hybridization or PCR probes.

Any number of additional, and conventionally employed, marker systems may be adapted to the method of this invention. One of skill understands that selection and/or implementation of a label system involves only routine experimentation. The labels and markers discussed above may be obtained commercially from known sources.

D. Solid Phase Capture Medium

The solid phase capture medium is typically a physiologically compatible bead, with any characteristic that allows it to be separated from the cell population of the sample. Such characteristics include refractive index, size, light scatter intensity (forward, side or 90°), or carrying a fluorescent detector dye to provide a unique fluorescent signature. Such beads are conventionally available in the art. For example, one subset of solid phase capture medium includes stable colloidal particles, such as polystyrene beads ranging in size from between about 0.2 to about 5.0 microns in diameter (i.e., colloidal-sized). Such polystyrene substrates or beads can contain aldehyde and/or sulfate functional groups, such as the commercially available beads, e.g., from Interfacial Dynamics Corporation, Portland, Oreg.

Alternatively, the polystyrene bead has an aminodextran coating over its peripheral surface and/or a colloidal-metal coating. Preferably an aminodextran coating is covalently bonded to the core substrate by covalent bonds between the free amino groups of the aminodextran and the amine-reactive functional groups of the polystyrene substrate and further by crosslinking with an agent such as glutaraldehyde. The aminodextran coating may generally be characterized as having a degree of diamine substitution in the range of 1/40-1/35 (1X-aminodextran) compared to a maximum theoretical value of 1/2.5. More preferably, the diamine substitution in the aminodextran coating is approximately 1/7 to 1/8 (5X-aminodextran). Analytes, particularly protein analytes, may be readily attached to these beads as is taught in the references cited below. See also, O. Siiman et al, “Covalently Bound Antibody on Polystyrene Latex Beads: Formation, Stability and Use in Analyses of White Blood Cell Populations”, J. Colloid Interface Sci., 233: (January 2001).

A variety of aminodextran beads are described in U.S. Pat. Nos. 6,074,884; 5,945,293; and 5,658,741. Aminodextran-coated monodispersed colloidal dispersions of magnetic ferrite [U.S. Pat. No. 5,240,640], metal [U.S. Pat. No. 5,248,772], polystyrene [U.S. Pat. No. 5,466,609; U.S. Pat. No. 5,707,877; U.S. Pat. No. 5,639,620; U.S. Pat. No. 5,776,706], and polystyrene-metal [U.S. Pat. No. 5,552,086; U.S. Pat. No. 5,527,713] particles may also be employed as formed bodies according to this invention.

Another type of bead may contain the above-described coated substrate with a layer of colloidal-sized metallic solid overlaying the aminodextran coating. Preferably this layer is uniformly dispersed over the dispersed surface of the aminodextran layer. The colloidal metal useful in forming the coated substrate is generally described as a metal which can be reduced from the ionic state to the metal(0) state by the aminodextran coating, or a metal which can form metal ions or metal ion complexes which have a reduction potential of about +0.7 volts or higher. While such metal ions may include: Ag(I), Au(III), Pd(II), Pt(II), Rh(III), Ir(III), Ru(II), Os(II), the preferred metal ions for such use are colloidal gold(III) and colloidal silver(I). Specifically, gold/silver colloid coated polystyrene-aminodextran beads, their preparation, characterization and use in analyses of subpopulations of white blood cells in whole blood have been described. See, e.g., U.S. Pat. No. 5,248,772; U.S. Pat. No. 5,552,086; U.S. Pat. No. 5,945,293; and O. Siiman and A. Burshteyn, 2000 J. Phys. Chem., 104:9795-9810; and O. Siiman et al, 2000 Cytometry, 41:298-307.

An alternative to this coated bead employs carboxy-functionalized polystyrene particles as the core substrate, coated with aminodextran by EDAC coupling as described in U.S. Pat. No. 5,639,620.

Other suitable beads that may be utilized in the methods of this invention are colored latex microparticles (Bangs Laboratories, Indiana) in which a dye is embedded and may be used to form complexes with the target, analyte or ligands. These beads also provide a visual signal indicative of the presence of the resulting complex in applicable assays. Still other suitable beads include nanocrystals, quantum dots and similar materials.

In one embodiment the bead is from 0.05 to 20 microns in diameter. In another embodiment, the bead is from 5 to 7 microns. In still another embodiment, the capture medium is greater than 1 μM in size. Mixtures of a variety of sizes of beads may also be employed, particularly where there are more than one soluble analyte to be detected. Generally, bead size impacts the sensitivity range of the assay, because smaller beads bind less antibody (see e.g., Lindmo, cited above or Frengen cited above). Therefore, in one embodiment, in which high sensitivity is required, a smaller number of larger beads is desirable for the assays. In the presence of large numbers of soluble analytes, a higher number of beads (both large and small) may be employed in these methods. For use in some embodiments of the present invention, the capture medium or bead is larger than the soluble analyte to be detected.

The capture medium may have bound thereto multiple ligands or multiple competing analytes. Each ligand bound to the capture medium is capable of binding to a soluble analyte or binding to an antibody that is itself capable of binding to the soluble analyte. Each competing analyte bound to the capture medium is capable of binding to a ligand (e.g., an antibody) that is capable of binding to the soluble analyte (whether labeled or unlabeled). Such ligands or competing analytes are associated or immobilized on the capture medium by conventional methods. For example, ligands or analytes such as antibodies, antigens, or linkers (e.g. Streptavidin, Protein A) may be attached to beads depending upon format of the analyte assay (competitive, immune-complex or sandwich) as described below. The beads may also be associated with detectable labels, preferably fluorescent labels, such as discussed above. Methods for attachment with such labels are disclosed in the texts cited herein.

Beads may be fluorescent or non-fluorescent, may be of different sizes or different fluorescent intensities, or both, for differentiation of multiple analytes. If using fluorescent intensity for labeling beads, it is preferred that the fluorescence emission should be unique for each population directed to a different analyte. Bead populations of different intensity are preferably resolvable if fluorescence of the bead is used as the only detectable label for discriminating among the soluble analyte and cellular target. Alternatively, if the size of the bead populations is used as the sole detectable label for discrimination among the soluble analyte and cellular target, each bead population must have a different forward scatter (FS) or side scatter (SS) than the cell population of interest in the assay.

Where the resulting analytic steps involve flow cytometry, the minimal parameters or characteristics of the beads are scatter (FS and/or SS) and at least two fluorescent wavelengths.

The relative volumes of the bead used in the sample container of the methods described herein are dependent upon bead concentration, analyte detection limits, and the cellular target, and sample size. For example, in one embodiment about 10 μL beads may be added to per 50-100 μL blood for 12×75 test tubes vs. 5 μL beads for 25-50 μL blood for microplate assays.

Preferably and optionally, solutions of bead populations useful in the present invention include a reagent that inhibits phagocytosis of the capture medium without damaging the target cells or inhibiting binding the target cells and the ligands.

Additionally or alternatively, the bead solution may contain an anti-coagulant, such as those mentioned below. Further the bead solutions may be kept at a temperature below 37° C., and more preferably, below 25° C., prior to addition to the sample or when introduced into the sample. These alternative and optional steps are also useful for inhibiting phagocytosis of the beads when in the sample.

E. Assay Formats

This method may utilize any number of conventional assay formats, for example, sandwich assays, competitive inhibition assays, immune complex assays, or others. Some of the components of these assays, as well as the conditions under which the sample is incubated, and the inclusion of optional steps or reagents, are dependent upon the assay selected. However, surprisingly, these assays using both beads and cellular markers provide accurate results in a single analysis. There is no negative effect on the binding of the beads in the presence of the cellular markers or vice versa. Surprisingly, there is no effect on the measurement of light scatter of fluorescent properties of the cells in the presence of the beads.

(1) Sandwich Assay

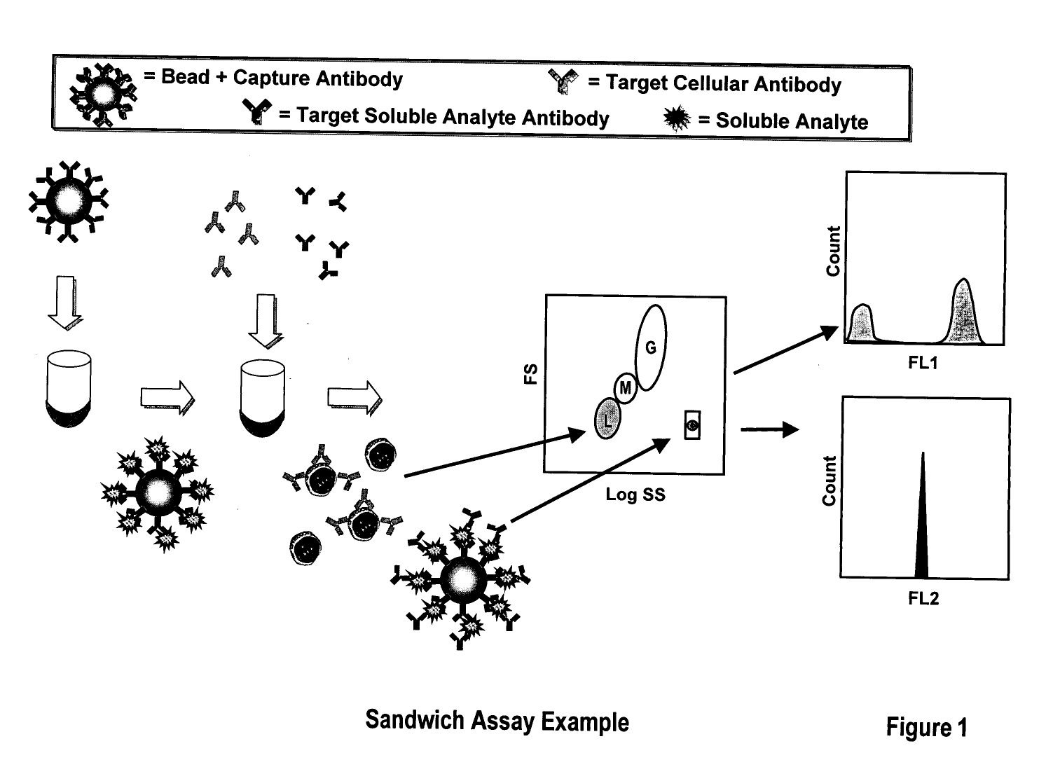

In one embodiment, the method can include the following steps for a sandwich assay. See, e.g., FIG. 1. A sample is introduced into a container, such microtiter plate wells or test tubes. The solid phase capture medium is added to the container. In this assay, the capture medium or bead has immobilized thereon multiple first ligands that are capable of binding the soluble analyte. Preferably the method employs vortexing, and incubation at a temperature of 37° C. or lower for about 5 minutes to up to 3 hours, and preferably for about 60 minutes. In one embodiment, the temperature is desirable lower than 25° C. or 22° C. The temperature and incubation times can be selected by one of skill in the art based upon the analyte, the analyte detection limit and the identity of the cellular target in the sample.

Thus after incubation and occasional mixing, a “first” complex is formed in the sample which consists of the capture medium, multiple immobilized first ligands, and multiple soluble analyte now bound to the capture medium by the first ligands.

An optional washing step may be employed before addition of the following components, depending upon required assay sensitivity. In some embodiments of these methods, a wash step to eliminate unbound first ligands is required for increased sensitivity.

Thereafter, suitable concentrations of at least two additional ligands are added to the sample. One of the additional ligands is a soluble second ligand smaller than the cellular target. The second ligand is capable of binding to the cellular target, e.g., to a cell surface or intracellular moiety. For example, an antibody to the cell surface antigen is a suitable ligand here. Each second ligand is desirably associated with a detectable label such as described above, and multiple second ligands can bind to a single target cell. The other of the additional ligands is a third ligand that is capable of binding to the soluble analyte whether that analyte be immobilized on the capture medium in the first complex or remaining soluble in the sample. This third ligand is desirably associated with a second detectable label that is different from the detectable label of the second ligand, i.e., the ligand that binds the cellular target.

In certain embodiments of this sandwich assay method, there are more than one second ligand directed to more than one target on the same cell type (e.g., an anti-CD45-PC5 antibody to the cell surface antigen CD45 and an anti-CD14-FITC antibody to the cell surface antigen CD14). In still other embodiments, more than one soluble ligand is directed to the same or to different targets on the same or different cell types. In certain embodiments of this assay more than one ligand is employed to more than one soluble analyte (e.g., an anti-IL-2-PE antibody to the soluble analyte IL-2, an anti-IL-6-PC7 antibody to the soluble analyte IL-6). Alternatively, more than one soluble ligand may be used for the same soluble analyte.

After these components are added to the sample, the sample is vortexed, and incubated with occasional mixing as described above. Thus the sample now contains a second complex consisting of the second labeled ligand(s) bound now to the cellular target(s) and a third complex comprising the third ligand bound to the soluble analyte which is bound through the first ligand to the capture medium. There may also be some small soluble third ligand-soluble analyte complexes in the sample.

Another optional step may be inserted into the assay method at this point, if the sample contains nucleated cells, such as red blood cells or NRBCs, and if higher sensitivity is needed for the analysis steps below. The sample may optionally be treated with an agent to lyse the nucleated cells. Another optional wash step may also be included to remove the lysed materials from the complexes., depending upon required assay sensitivity.

The final step of this method is a simultaneously analysis of the sample treated as described above, without physically separating the various complexes to be measured. Given the above steps of this method, one may taken the sample containing these complexes and discriminate between the third complex comprising the third ligand bound to the soluble analyte which is bound through the first ligand to the capture medium and the second complex consisting of the second labeled ligand(s) bound now to the cellular target(s) using the same sample. Methods suitable for performing this analysis step include image analysis and, preferably, flow cytometric analysis. A flow cytometric analysis is conducted by employing a gating strategy appropriate to the sample type. For example, the third complex containing the beads is gated separately from the second complex of the ligand-labeled cells based on light scatter and fluorescence intensity. Thereafter, if more than one fluorescent label is present on the cell target or the bead, the strategy can provide separate compensation for each fluorophore. Similarly other cell parameters, such as differentially expressed targets and intracellular targets may also be measured during this analysis.

The standards for quantitation of the analyte include cell controls with serum-based analyte standards. Such standards are applicable to all three assay types described herein. These standards are stabilized cells in a media containing the soluble analytes of interest.

(2) Competitive Inhibition Assay

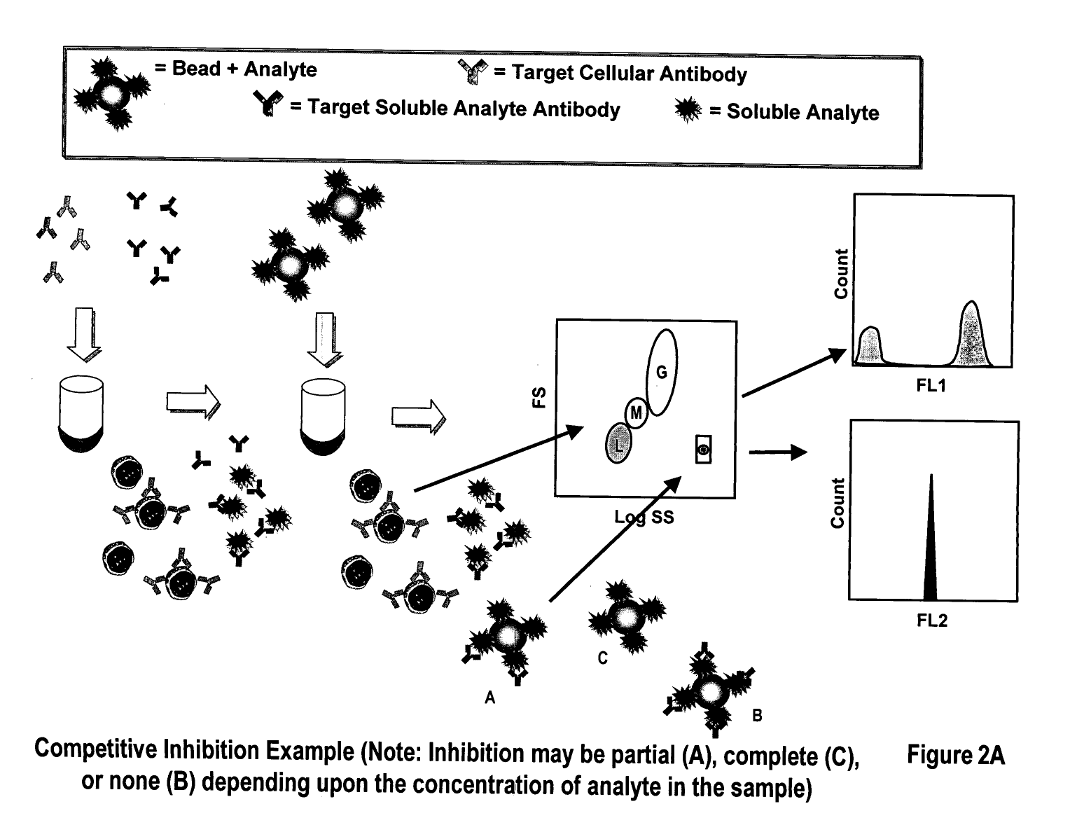

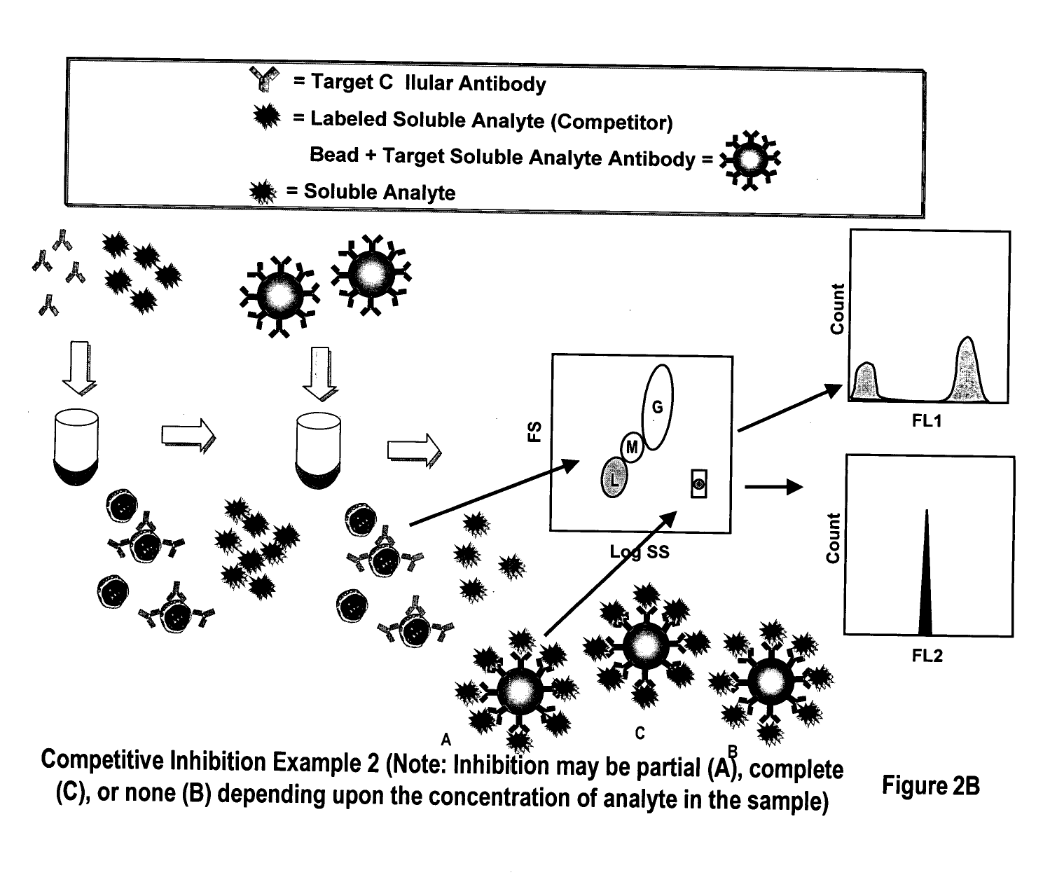

In still other embodiments, the method can include the following steps for a competitive inhibition assay. See, e.g., FIG. 2A and FIG. 2B.

In one format depicted in FIG. 2A, a sample is introduced into a container, such microtiter plate wells or test tubes. A known concentration of a first soluble ligand capable of binding to a single cellular target is added to the sample. This first ligand is desirably associated with a first detectable label. Multiple of the first ligands may bind to the cell. At the same time a known concentration of a second ligand capable of binding the soluble analyte is added to the sample. The second ligand is associated with a second detectable label. After vortexing and incubating for from about 5 minutes to about 3 hours, preferably up to 60 minutes, at a temperature of under 37° C., a first complex is formed which includes the cellular target bound to the first labeled ligand and a second complex is formed comprising soluble analyte bound to the second labeled ligand.

Thereafter a solid phase capture medium on which are immobilized a known multiple of the same analytes is added to the sample. The sample is vortexed and incubated again under the same conditions, and a third complex is formed consisting of the capture medium, the analyte immobilized thereof and any of the second ligand in the sample that did not bind to the soluble analyte.

An optional washing step may be employed after the addition of the components, depending upon required assay sensitivity.

Another optional step may be inserted into the assay method at this point, if the sample contains nucleated cells, such as red blood cells or NRBCs, and if higher sensitivity is needed for the analysis steps below. The sample may optionally be treated with an agent to lyse the nucleated cells. Among such agents are included without limitation, ImmunoPrep reagents (Beckman Coulter), ammonium chloride, etc. Another optional wash step may also be included to remove the lysed materials from the complexes, depending upon required assay sensitivity.

The final step of this method is a simultaneous analysis of the sample treated as described above, without physically separating the various complexes to be measured. Given the above steps of this method, one may take the sample containing these complexes and discriminate between the first complex comprising the cellular target bound to the first labeled ligand and the third complex consisting of the capture medium, the analyte immobilized thereof and any of the second ligand in the sample that did not bind to the soluble analyte. The amount of third complex detected is proportional to the amount of soluble analyte present in the sample.

In one format depicted in FIG. 2B, a sample is introduced into a container, such microtiter plate wells or test tubes. A known concentration of a first soluble ligand capable of binding to a single cellular target is added to the sample. This first ligand is desirably associated with a first detectable label. Multiple of the first ligands may bind to the cell. At the same time a known concentration of a competing soluble analyte is added to the sample. The competing soluble analyte is preferably associated with a second detectable label. After vortexing and incubating for from about 5 minutes to about 3 hours, preferably up to 60 minutes, at a temperature of under 37° C., a first complex is formed which includes the cellular target bound to the first labeled ligand.

Thereafter a solid phase capture medium on which are immobilized a known multiple of a ligand that binds to the soluble analyte (competing analyte or naturally occurring analyte in the sample, if any) is added to the sample. The sample is vortexed and incubated again under the same conditions, and potential second and third complexes are formed. A second complex is formed by the capture medium-immobilized ligand and the naturally occurring soluble analyte in the sample, if any (unlabeled). A third complex is formed by any of the capture medium-immobilized ligand that did not bind to the unlabeled soluble analyte and the competing analyte (labeled). No complex is formed between the competing, labeled soluble analyte and the unlabeled soluble analyte occurring naturally in the sample.

An optional washing step may be employed after the addition of the components, depending upon required assay sensitivity.

Another optional step may be inserted into the assay method at this point, if the sample contains nucleated cells, such as red blood cells or NRBCs, and if higher sensitivity is needed for the analysis steps below. The sample may optionally be treated with an agent to lyse the nucleated cells. Among such agents are included without limitation, ImmunoPrep™ reagents (Beckman Coulter), ammonium chloride, etc. Another optional wash step may also be included to remove the lysed materials from the complexes, depending upon required assay sensitivity.

The final step of this method is a simultaneous analysis of the sample treated as described above, without physically separating the various complexes to be measured. Given the above steps of this method, one may take the sample containing these complexes and discriminate between the first complex comprising the cellular target bound to the first labeled ligand and the third complex consisting of the capture medium-immobilized ligand and competing analyte (labeled). Additionally the second complex of the capture medium with the unlabeled analyte may also be detected. The amount of third complex detected is proportional to the amount of soluble analyte (unlabeled) present in the sample.

As with the sandwich assay, one may manipulate this assay for measurement of more than one cell type, more than one cellular target on a cell type, or more than one soluble analyte by selecting from among any number of soluble ligands, detectable labels, and solid phase capture media on which is immobilized different ligands or competing analytes. Methods suitable for performing the analysis step include image analysis and, preferably, flow cytometric analysis. A flow cytometric analysis is conducted by employing a gating strategy appropriate to the sample type. For example, the complexes containing the beads are gated separately from the complex of the ligand-labeled cells based on light scatter and fluorescence intensity. Thereafter, if more than one fluorescent label is present on the cell target or the bead, the strategy can provide separate compensation for each fluorophore. Similarly other cell parameters, such as differential and intracellular antigens or other targets may also be measured during this analysis.

(3) Immune Complex Assay

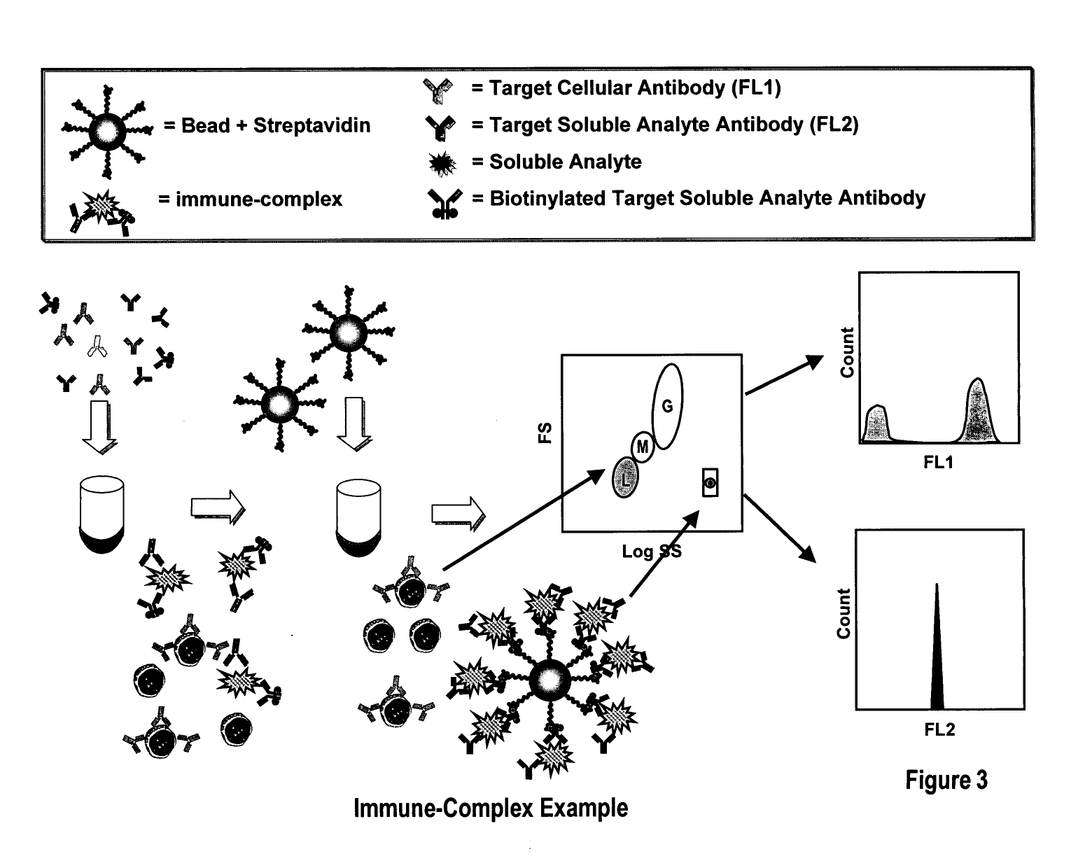

In one embodiment, the method can include the following steps for an immune complex assay. See, e.g., FIG. 3. A sample is introduced into a container, such as a microtiter plate well or test tube. A first soluble ligand capable of binding to the cellular target is added to the sample. Multiple of these first ligands may bind a single target cell. Desirably these first ligands provide a first detectable signal, preferably due to association with a detectable label. Also added to the sample is a second ligand, capable of binding to the soluble analyte. This second ligand is also preferably labeled and can provide a second detectable signal. To the same sample is added a third ligand capable of binding to the same soluble analyte, which ligand is associated with a different detectable label. After vortexing and incubating for from about 5 minutes to about 3 hours, preferably up to 60 minutes, at a temperature of under 37° C., a first complex is formed comprising the first cellular target and the first ligand and a second complex is formed comprising the soluble analyte bound to one or both of the second ligand and third ligand.

Thereafter, a solid phase capture medium on which is immobilized multiple fourth ligands is added to the sample. These fourth ligands are capable of binding to the second or third ligands. After vortexing, and incubating under the conditions described above, a third complex is formed. This third complex consists of which the solid phase capture medium bound to multiple fourth ligands, with each fourth ligand bound to a third ligand. Each third ligand is also bound to a soluble analyte, which is then further bound to one or more second ligands.

An optional washing step may be employed after the addition of the assay components, depending upon required assay sensitivity. Another optional step may be inserted into the assay method at this point, if the sample contains nucleated cells, such as red blood cells or NRBCs, and if higher sensitivity is needed for the analysis steps below. The sample may optionally be treated with an agent to lyse the nucleated cells. Among such agents are included without limitation, ImmunoPrep reagents (Beckman Coulter), ammonium chloride, etc. Another optional wash step may also be included to remove the lysed materials from the complexes, depending upon required assay sensitivity.

The final step of this method is a simultaneous analysis of the sample treated as described above, without physically separating the various complexes to be measured. Given the above steps of this method, one may take the sample containing these complexes and discriminate between the first complex, the second complex and the third complex.

As with the sandwich assay, one may manipulate this assay for measurement of more than one cell type, more than one cellular target on a cell type, or more than one soluble analyte by selecting from among any number of soluble ligands, detectable labels, and solid phase capture media on which is immobilized different analytes. Methods suitable for performing the analysis step include image analysis and, preferably, flow cytometric analysis. A flow cytometric analysis is conducted by employing a gating strategy appropriate to the sample type. For example, the complex containing the beads is gated separately from the complex of the ligand-labeled cells based on light scatter and fluorescence intensity. Thereafter, if more than one fluorescent label is present on the cell target or the bead, the strategy can provide separate compensation for each fluorophore. Similarly other cell parameters, such as differential and intracellular targets or antigens may also be measured during this analysis.

F. Optional Method Steps

The methods of this invention can also include a number of optional steps.

(1) Washing Steps

For example, where increased sensitivity of the assays are desirable, washing steps with buffer, or diluent can be introduced into the methods. Generally, such washing steps can be introduced after the incubation of the sample with the capture medium to eliminate materials not bound to the capture medium. Alternatively, such washing steps can follow incubation with soluble ligand to eliminate uncomplexed materials. Still another option includes washing the sample after an optional lysis step to rid the sample of lysed RBC components.

(2) Inhibiting Phagocytosis

Another optional step suitable for the methods of this invention is the addition of a reagent that inhibits phagocytosis of the capture medium by cells, particularly myeloid cells in the sample, without damaging the target cells or inhibiting binding the target cells and the ligands used in the methods. A suitable phagocytosis inhibitor is sodium azide (preferably, at a concentration of less than 0.01% v/v). Gliotoxin, gliotoxin-trisulfide and gliotoxin-tetrasulfide and related compounds belonging to the class of epipolythiodioxopiperazines also inhibit phagocytosis by macrophages, white cells that participate in the host's defense system. See, e.g., U.S. Pat. No. 4,886,796. Another suitable phagocytosis inhibitor is cytochalasin B (see also, U.S. Pat. No. 5,162,990). Other phagocytosis inhibitors include protein kinase inhibitors, an excess of heavy metals such as zinc, cadmium, lead, mercury, etc., phosphatase inhibitors such as pyrophosphate and levamisole, an excess of adenosine or the polyamines putrescine and spermidine, cycloheximide, EDTA, bromoenol lactone, and other phospholipase inhibitors and cytochalasin D.

The phagocytosis inhibitors may be added to the bead solutions particularly when the biological samples contain myeloid cells, because phagocytosis of beads by myeloid cells is common. While the phagocytosis inhibiting reagent may be added to the capture medium prior to addition of the capture medium to the sample, it is also possible to add the phagocytosis inhibiting reagent to the capture medium at the same time the capture medium is added to the sample. Alternatively, the phagocytosis inhibiting reagent is added to the sample prior to addition of the capture medium to the sample. The phagocytosis inhibiting reagent is added to the sample at the same time the capture medium is added to the sample. In one embodiment, it has been determined that where the method employs beads less than 5 [m in diameter, it is preferable to introduce multiple phagocytic inhibitors, such as combinations of the inhibitors identified above.

(3) Lysis

Another optional step of the method for samples that contain blood cells includes lysing the sample to remove the generally very numerous nucleated blood cells, including RBC and NRBC prior to the analyzing step. Lysing agents, preferably detergents, more preferably nonionic detergents, are used to break down cell membranes, thus releasing DNA, RNA and proteins from the cells. Any suitable lysing agent may be employed. Buffered halides, such as ammonium chloride and Trizma based (e.g., about 7.5 g ammonium chloride and 2 g Tris per liter), define one suitable class of lysing agents, where the undesired cells include red blood cells. Lysing is accomplished through a series of consecutive washing steps with the lysing agent. Optionally, before the lysing, the cells are subjected to a preliminary fixing step, such as by contacting them with a suitable fixing agent, heating them or both. For instance, the cells are contacted with a buffered antimicrobial saline solution including a suitable amount of a fixative (e.g., about 0.11% formaldehyde).

Still other lytic agents are included without limitation, ImmunoPrep™ reagents (Beckman Coulter), ammonium chloride, etc. In one embodiment, a lytic reagent is Bacterial Protein Extraction Reagent (BPER), a proprietary mixture of nonionic detergents marketed by the Pierce Chemical Company. Other nonionic detergents are useful and many detergents are operable, even some anionic and cationic detergents under certain applications. The nonionic detergent lysing agents are generally be added to the sample in a concentration of about 0.1 to 5, more preferably 0.5 to 2 wt %. Other known lysing agents can also be used with the technology such as freeze/thawing, French cell press, enzymes, microfluidization, sonication, etc.

As stated above, the sample may then be optionally washed after lysis.

(4) Adding an Inhibitor of Cellular Activation

Still another optional step that can be included in the methods described herein includes contacting the sample with an inhibitor of cellular activation. The inhibitor of cellular activation is contacted with the sample prior to or simultaneously with the addition to the sample of the capture medium or ligands used in the methods. The inhibitor of cellular activation can be one or more of an anticoagulant, an inhibiting reagent, a fixative or an inhibiting reaction temperature.

(a) Anticoagulants

Anticoagulation of the sample can be accomplished by binding or chelation of calcium ions by a variety of substances. Conventional anticoagulants include, without limitation, ethylenediaminetetraacetic acid (EDTA) or a salt thereof, a citrate salt of sodium or potassium, an oxalate salt of sodium or potassium, or combinations thereof. Other traditional anticoagulants include natural enzymatic inhibitors of the coagulation sequence, such as heparin or sodium fluoride or hirudin. Still other anticoagulants include, without limitation, protease, protein kinase inhibitors such as phenylmethylsulfonylfluoride (PMSF), 4-(2-aminoethyl) benzenesulfonyl- fluoride (AEBSF), tosyl-lysine chloro-methyl ketone (TLCK), tosyl-phenylalanine chloromethyl ketone (TPCK), leupeptin, epstatin A, 1-(5-isoquinolinesulfonyl) piperazine. Such anticoagulants or preservatives may be used alone or in combination for addition to the sample. See, for example, U.S. Pat. Nos. 5,935,857 and 4,528,274. Anticoagulants may be added to the sample in this invention preferably prior to the addition of the ligands and/or capture medium.

(b) Fixatives

Another optional step to be added to the methods above includes the addition of a fixative to the sample prior to the introduction of the ligands or capture medium. “Fixatives” include formaldehyde, paraformaldehyde, and glutaraldehyde, dehydrating alcohols, glyoxal, and organic acids, such as acetic acid, formic acid, and picric acid, mercuric compounds, tannic acid and many other compounds. Another useful fixative is described in U.S. Pat. No. 5,459,073 which fixative has low toxicity employing a formaldehyde donor, such as diazolidinyl urea, imidazolidinyl urea, dimethylol-5,5-dimethylhydantoin, dimethylol urea and the like rather than formaldehyde itself.

(c) Inhibiting Reagents

Still another optional step is to add to the sample an inhibiting reagent to control cellular activation. Suitable reagent compositions can include one or more protease inhibitor(s). A non-exclusive list of protease inhibitors for use in the present invention includes the serine protease inhibitors, such as 4-(2-aminoethyl)benzenesulfonyl fluoride hydrochloride (AEBSF), which has a molecular weight of 230.7 and inhibits catalytic activity of the protease active site; antithrombin plasma protein (60,000 MW) that inhibits thrombin and other serine proteases in the blood clotting cascade; or 4-amidinophenylmethanesulfonyl-fluoride-HCl (APMSF, 352.7 MW), an irreversible inhibitor of trypsin-like serine proteases. Still other serine proteases include Aprotinin (6500 MW) that inhibits serine proteases by tightly binding to the active site of the enzyme; diisopropyl phosphorofluoridate (DFP, 184.2 MW), a very toxic, irreversible inhibitors of serine proteases and acetylcholine esterase; phenylmethanesulfonyl fluoride (PMSF, 174.2 MW), which is another toxic, irreversible inhibitor that acts by chemically modifying the active site of the enzyme; and α-toluenesulfonyl fluoride.

Other suitable serine and cysteine protease inhibitors useful in the methods of this invention include antipain (678.2 MW), a reversible inhibitor of proteasese and of RNA synthesis; chymostatin (600 MW), a reversible inhibitor of some serine and cysteine proteases; leupeptin (475.6 MW) a reversible competitive inhibitor of trypsin-like proteases; L-1-chloro-3-[4-tosyl-amido]-7-amino-2-heptanone-HCl (TLCK, 369.3 MW), which inhibits irreversibly by chemically altering the enzyme active site; and L-1-chloro-3-[4-tosylamido]-4-phenyl-2-butanone (TPCK, 351.8), which irreversibly inhibits by chemically altering the enzyme active site.

Still other suitable cysteine protease inhibitors useful in this invention include E-64 (357.4 MW), a non-competitive irreversible inhibitor of cystein proteases. Other suitable protease inhibitors inhibit metaloproteases. For example, amastatin (511MW) is a non-toxic reversible inhibitor; bestsatin (244.8 MW) is a multi-function metallo-protease inhibitor that has anticarcinogenic and immunomodulating properties; diprotin (341.5 MW), a reversible inhibitor; EDTA (372.3 MW) a reversible inhibitor that acts by chelating enzyme cofactors and may interfere with other metal dependent biological processes. Other metaloprotease inhibitors include vanadium, molybdate salts, and 1,10-phenanthroline. Still other suitable inhibitors for use in this invention are aspartic protease inhibitors, such as pepstatin (685.9 MW) a peptide that provides reversible inhibition.

In one embodiment, the methods above include the step of introducing into the sample a single inhibitor. In another embodiment, the invention includes adding combinations of two or more such inhibitors, to permit use of small amounts of those inhibitors that are toxic or cause otherwise undesirable effects if used alone in large concentrations. It is desirable for the concentration of protease inhibitor(s) in the stabilizing reagent composition to be up to about 10 mM. However, the range of concentrations is entirely dependent upon the inhibitor(s) used. This range is determined based upon the experimental data of inhibition of platelet activation, as described herein. One of skill in the art given the teachings provided herein would readily be able to determine, with only a minimal and conventional amount of experimentation, a desirable concentration for each specific inhibitor used in the assay methods.

Another group of useful inhibitors includes one or more phosphatase inhibitor(s). A non-exclusive list of suitable phosphatase inhibitors includes, without limitation, pyrophosphate, microcystin LA, microcystin LR, tetramisole, L-4-bromotetramisole, tautomycin, okadaic acid, calyculin, thrysiferyl-23-acetate, cantharidine, vanadium salts, sodium orthovanadate, tartrate salts, phloridzin, molybdate salts, and imidazole. For other suitable inhibitors, see Handbook of Enzyme Inhibitors, Melmward Sollner (1989), ISBN 3-527-26994-0; ISBN 0-89537-860-0, incorporated by reference herein. In one embodiment, the methods of this invention include adding a single phosphatase inhibitor to the sample. In another embodiment, the methods of this invention include adding combinations of two or more such inhibitors, to permit use of small amounts of those inhibitors that are toxic or cause otherwise undesirable effects if used alone in large concentrations. It is desirable for the concentration of a phosphatase inhibitor(s) in the sample to be up to about 120 mM. However, the range of concentrations is entirely dependent upon the inhibitor(s) used. This range is determined based upon the experimental data of inhibition of platelet activation, as described herein. One of skill in the art given the teachings provided herein would readily be able to determine, with only a minimal and conventional amount of experimentation, a desirable concentration for each sample.

(d) Temperature

Another optional step of the present invention useful for inhibiting cellular activation and making the processes of this invention more efficient is the use of inhibiting reaction temperatures in the method of below 25° C. Preferably, such lower temperature incubations can occur at a temperature of between 4° C. and 25° C. In one embodiment, the temperature is below 20° C. In another embodiment, the temperature is below 15° C. In still another embodiment, the temperature is below 10° C. In still another embodiment, the temperature is below 7° C. One of skill in the art given the disclosures herein may readily select the appropriate temperature for the method employed.

G. Specific Methods of the Invention

The methods of the present invention are useful in diagnosis of a variety of mammalian diseases or conditions. Examples of such diseases or conditions include, without limitation, sepsis, inflammation, autoimmune disease, cardiovascular disease, viral infection, bacterial infection, cancer, and drug activities, half-life, or interactions. Exemplary drugs include thyopolin, insulin, and chemotherapeutics. The methods of the present invention are also useful for evaluation of food or water or other products for contamination with microorganisms or toxins or other contaminants.

In one embodiment, the above described assay methods of this invention are useful in a method for diagnosing sepsis or monitoring the progress thereof. This method is accomplished by performing the desired assay method above with soluble ligands that bind cellular targets including, but not limited to CD64 (N), HLA-DR (Mo), CD11a, CD14/Cd16, and CD142 (tissue factor) and using soluble ligands and capture medium that bind directly or indirectly the soluble analyte, which may be one or more of IL-6, IL-10, IL-1, TNF-∀, neopterin, C-reactive protein, procalcitonin, or activated Protein C.

In another embodiment, the methods above may be adapted for use in diagnosing autoimmune disease or monitoring the progress thereof. According to this aspect of the invention, the assay methods above employ ligands that bind one or more of the cell types including activated T cells and activated B cells by one or more of the cell surface or intracellular antigens that characterize those cells. The methods also use the ligands and capture medium to bind a soluble analyte, which may be one or more of C-reactive protein, a chemokine, or a cytokine. The selection of chemokine or cytokine used as the soluble reagent may be readily made by one of skill in the art.

In still a further embodiment, the methods of this invention are useful in diagnosing cardiovascular disease or monitoring the progress thereof. Such methods employ as the cellular target one or more of platelet-leucocyte aggregates, or CD142 (TF) and use ligands that bind thereto. This method is useful in also targeting the soluble analyte, which may be hsC-reactive protein, troponin, or myoglobin. Suitable ligands and capture medium for use in this method may be designed and selected by one of skill in the art given this disclosure.

A method for differential diagnosis of viral and bacterial infections or for use in monitoring the progress thereof employs the assay steps disclosed herein with ligands capable of binding a cellular target, which includes, without limitation, one or more of HLA-DR, CD4/CD8, CD64(N), or CD14/CD16. The soluble analyte, which may be one or more of IFNγ, neopterin, or C-reactive protein is detected by ligands and capture medium that bind directly or indirectly these analytes.

In still another embodiment, the methods of the invention are suitable for detecting and monitoring contaminants in fluid, such as water systems, or other liquid products. For example, water may be examined for the presence of bacterial cells by using ligands to cell surface antigens or intracellular antigens of bacterial origin, and for soluble analytes, such as toxins, by using a capture medium on which is associated a legend to the toxin. For example, in one embodiment, the soluble analyte is an enterotoxin, such as cholera, and the cellular target is the enterococcus. One of skill in the art may select other examples of such pollutants and targets for suitable use in methods of this invention.

The methods and compositions of this invention are also adaptable to the diagnosis and monitoring of other diseases and conditions, based on the identification of cellular targets, soluble targets and ligands that bind thereto, as directed by this specification.

These methods, which allow for simultaneous analysis of relevant cellular and soluble targets, cellular antigens, cell characteristics and hematology parameters, provide a more complete picture than do prior art methods of a patient's medical status with regard to both cellular and soluble mediators, activators or inhibitors. This invention permits multiple assays to be conducted in a single test tube or microtiter plate and allows a comprehensive snapshot of patient status or drug effects. The advantages of the methods of this invention include decreased sample size (e.g., blood) requirements, which are particularly important for pediatric and geriatric patients, increased accuracy or clinical monitoring, increased throughput efficiency, reduced time and labor to conduct the tests, and decreased overall cost to a patient. For example, the ability to assess activated immune cells in combination with a variety of soluble analytes can improve both the diagnosis and monitoring of the above-noted diseases.

H. Kits

For convenience, the conventional reagents for high throughput assays or other diagnostic assays useful according to this invention may be provided in the form of kits. In yet another aspect of this invention, a kit is provided for performance of the above-described methods. Preferably such kits are employed for performing the diagnostic methods of this invention and/or monitoring therapy. However, such kits can be assembled for research purposes also. Thus, a kit of the present invention desirably contains the components taught above, e.g., at least one soluble ligand that binds a cellular target in the sample; at least one soluble ligand that binds a soluble analyte in the sample or at least one competing soluble analyte (preferably labeled); and a solid phase capture medium that binds directly to the soluble analyte, indirectly to the soluble analyte, or to the soluble ligand that binds to the soluble analyte. The kits also include instructions for performing the particular assay, various diluents and buffers, and signal-generating reagents, such as fluorophores, enzyme substrates, cofactors and chromogens. Other components may include indicator charts for colorimetric comparisons, disposable gloves, decontamination instructions, applicator sticks or containers, and a sample preparator cup.

In one embodiment of the present invention, a kit useful for the performance of the above-described sandwich assay includes, as a component, a solid phase capture medium associated with multiple first ligands that bind the soluble analyte. Another kit component is the soluble ligand that binds the cellular target and is associated with a first detectable label. The kit further comprises a third ligand that is capable of binding to the soluble analyte-first ligand-capture medium complex. The third ligand associated with a second detectable label.

In another embodiment, a kit for performing one of the competitive inhibition assays described above, contains a first ligand associated with a first label. Multiple of the first ligands are capable of binding to a single cellular target. Another component is a second ligand associated with a second label. The second ligand is capable of binding a soluble analyte. Still another component is the solid phase capture medium associated with multiple of the soluble analytes immobilized thereon.

In another embodiment, a kit for performing another of the competitive inhibition assays described above, contains a first ligand associated with a first label. Multiple of the first ligands are capable of binding to a single cellular target. Another component is a competing analyte associated with a second label. Still another component is the solid phase capture medium on which are immobilized multiple of ligands capable of binding to the soluble analyte (either competing soluble analyte or soluble analyte naturally occurring in the sample).

In yet another embodiment, a kit for performing the immune complex assay of this invention includes a first ligand capable of binding to a first cellular target and providing a first detectable signal; a second ligand capable of binding to the soluble analyte and providing a second detectable signal; a third ligand capable of binding to the same soluble analyte; a solid phase capture medium on which is immobilized multiple fourth ligands, the fourth ligands capable of binding to the third ligands.

Such kits are useful for evaluating blood samples for purposes of determining disease states associated with inappropriate types or numbers of blood cells, blood cell types or bound components or soluble antigens or analytes thereof. Thus, such a kit will be useful in conducting the diagnostic assays discussed herein, e.g., in determining the status of treatment of an illness characterized by inappropriate cell target or soluble analyte expression in a blood sample. Such a diagnostic kit contains the dyes, ligands, capture medium, and other components of the methods of this invention. Such kits also contain labels, exemplified above, pre-attached to the other components of the specific assay to be performed, or provided separately for attachment to a selected component, e.g., a substrate. Alternatively, such kits may contain a simple mixture of such compositions or means for preparing a simple mixture.

Such kits provide a convenient, efficient way for a clinical laboratory to screen blood samples or other biological samples containing cells according to this invention.

One of skill in the art may be expected to vary the components of these diagnostic kits in obvious ways based on the knowledge in the art coupled with this disclosure. Such varied components are included in this embodiment of the invention.

The kit further comprising at least one of the following additional components selected from the group consisting of suitable vessels for containing samples, suitable controls or tables of normal or disease-characteristic values of activated platelets; an anti-coagulant or coagulation pathway inhibitor, other reagents suitable for the performance of flow cytometric analyses and combinations thereof, suitable diluents and buffers for the samples, disposable gloves, decontamination instructions, applicator sticks or containers, and sample preparator cups.

### EXAMPLES

These examples demonstrate the use of the methods and compositions of the invention and the analysis thereof. The data reported in these Examples demonstrates that the novel methods of this invention have performance parameters that permit improved efficient and simultaneous analysis of samples with multiple types of targets. These examples are illustrative and do not limit the scope thereon. One of skill in the art will appreciate that although specific reagents and conditions are outlined in the following examples, modifications as described above can be made to provide the compositions of this invention or processes for use thereof.

### Example 1

### Immuno-Plex Multi Analyte Sandwich Assay

To a sample (100 uL) of EDTA-treated whole blood are added the following reagents:
(a) The capture medium is 50 μl of a six-bead polystyrene microsphere bead population with distinct fluorescence intensities. The microspheres or beads are generally larger than 3.6 μm and smaller than 10 μm. Antibodies are individually covalently attached to a subset of the beads by conventional methods to create the antibody-conjugated fluorescence Capture Beads with bead specificity for IL-2, IL-4, IL-5, IL-10, TNF-α or IFNγ.
(b) Phycoerythrin (PE)- conjugated anti-human detector soluble ligand (50 ul/test) with an antibody specificity for anti-IL-2, anti-IL-4, anti-IL-5, anti-IL-10, anti-TNF-α, or anti-IFN-γ.
(c) A soluble ligand to the cellular target, i.e., 20 μl of anti-CD45-FITC (Beckman Coulter).

This reagent mixture is incubated for between about 1 hour to 3 hours at room temperature and also is protected from light.

Once incubated, the red blood cells in the sample are lysed by the addition of ImmunoPrep™ reagents (Beckman Coulter). The sample is then subjected to cytometric flow analysis without any further manipulation or separation of the various complexes formed among the reagents in the sample and the data collected.

### Example 2

### Immuno-Plex Single Analyte Capture Assay

A sample of whole blood (100 μl) collected in the anticoagulant EDTA, is kept on ice or at room temperature throughout this experiment. To this sample is added the following reagents:
(a) 10 μl of Capture Beads. As Capture Bead, a single non-fluorescence, paramagnetic, polystyrene microsphere population to which are bound antibodies to the soluble analyte IL2, i.e., human IL-2 antibodies. The beads are larger than 3.6 μm and smaller than 10 μm.
(b) 20 μl of a ligand to the cellular target CD14, which is an antibody labeled with fluorescent isothiocyanate (anti-CD14-FITC) (Beckman Coulter); and
(c) 10 μl of a ligand to the cellular target CD45, which is an antibody labeled with phycocyanin-5 (anti-CD45-PC5) (Beckman Coulter). The sample is then incubated for 60 minutes mixing twice every 30 seconds (or alternatively rocking).

Thereafter 10 ul of a soluble ligand to the soluble analyte IL-2, i.e., phycoerythrin (PE) conjugated anti-IL2 reporter antibody, is added to the sample and the sample is incubated for 30 minutes, again mixing twice per 30 seconds (or rocking).

The sample is then lysed using ImmunoPrep™ reagents (Beckman Coulter). The sample is then subjected to cytometric flow analysis without any further manipulation or separation of the various complexes formed among the reagents in the sample and the data collected.

All documents cited above are incorporated by reference herein. The compositions and processes of the present invention are encompassed by the scope of the claims appended hereto.

1. A method for evaluation of a biological sample containing at least one cell type bearing at least one cellular target and at least one soluble analyte, the steps comprising:
(a) adding to a single container said sample with
(i) at least one soluble ligand that binds to said cellular target,
(ii) at least one soluble ligand that binds to said soluble analyte, or at least one competing soluble analyte; and
(iii) a solid phase capture medium that binds directly to said soluble analyte, indirectly to said soluble analyte, or to said soluble ligand that binds to said soluble analyte; and
(b) simultaneously analyzing said sample (a) without physically separating the complexes comprising a complex that forms between said cellular target and said at least one soluble ligand, and a complex that forms between said capture medium bound directly to said soluble analyte, or between said capture medium bound indirectly to said soluble analyte, or between said capture medium bound to said soluble ligand that is bound to said soluble analyte.

2. The method according to claim 1, wherein step (a) further comprises the steps:
(a1) adding to said sample a solid phase capture medium on which are immobilized multiple first ligands that are capable of binding to said soluble analyte, and wherein said capture medium, said first ligand, and said soluble analyte form a first complex;
(a2) adding to said sample of step (al) a soluble second ligand that is capable of binding to said cellular target, wherein each second ligand is associated with a first detectable label, and wherein multiple second ligands can bind to a single target cell; wherein said second ligand and said cellular target form a second complex;
(a3) adding to said sample of step (a2) a third ligand that is capable of binding to said first complex, said third ligand associated with a second detectable label, wherein said third ligand and said first complex form a third complex; and
wherein step (b) comprises simultaneously analyzing said sample of step (a3) without physically separating said complexes by discriminating between said third complex and said second complex.

3. The method according to claim 1, wherein step (a) further comprises the steps:
(a1) adding to said sample (i) a known concentration of a first ligand associated with a first label, wherein multiple of said first ligands are capable of binding to a single said cellular target; and (ii) a known concentration of a second ligand associated with a second label, wherein said second ligand is capable of binding to said soluble analyte; and wherein said cellular target and said first labeled ligand form a first complex and said soluble analyte and said second labeled ligand form a second complex;
(a2) adding to said sample (al) a solid phase capture medium on which are immobilized a known multiple of said analytes, and wherein said capture medium with immobilized analyte and any of said labeled second ligand not bound to said soluble analyte in said sample form a third complex; and
wherein step (b) comprises simultaneously analyzing said sample (a2) without physically separating said complexes by discriminating between said first complex and said third complex, wherein the amount of third complex detected is proportional to the amount of soluble analyte present in said sample.

4. The method according to claim 1, wherein step (a) further comprises the step:
(a1) adding to said sample (i) a known concentration of a first ligand associated with a first label, wherein multiple of said first ligands are capable of binding to a single said cellular target; and (ii) a known concentration of a competing soluble analyte associated with a second label, and (iii) a solid phase capture medium on which are immobilized a known multiple of second ligands capable of binding to said soluble analyte,
wherein said cellular target and said first labeled ligand form a first complex; wherein said labeled competing soluble analyte and said unlabeled soluble analyte in said sample compete for binding to said capture medium with immobilized second ligand, thereby forming a second complex wherein said capture medium is bound to labeled competing analyte and a third complex wherein said capture medium is bound to unlabeled soluble analyte; and
wherein step (b) comprises simultaneously analyzing said sample (al) without physically separating said complexes by discriminating between said first complex and said second complex, wherein the amount of second complex detected is proportional to the amount of unlabeled soluble analyte present in said sample.

5. The method according to claim 1, wherein step (a) further comprises the steps:
(a1) adding to said sample (i) a first ligand, wherein multiples of said first ligand are capable of binding to said cellular target and providing a first detectable signal; (ii) a second ligand, capable of binding to said soluble analyte and providing a second detectable signal; and (iii) a third ligand capable of binding to the same said soluble analyte; wherein said first cellular target and said first ligand form a first complex, wherein said soluble analyte bound to one or both of said second ligand and third ligand form a second complex;.
(a2) adding to said sample (al) a solid phase capture medium on which is immobilized multiple fourth ligands, said fourth ligands capable of binding to said second or third ligands, wherein said solid phase capture medium with immobilized fourth ligands, and said third ligands bound to a soluble analyte, which is bound to one or more second ligands form said third complex; and
wherein step (b) further comprises simultaneously analyzing said sample (a2) without physically separating said complexes by discriminating among said first complex, said second complex and said third complex.

6. The method according to claim 1, wherein said at least one soluble ligand (i) comprises multiple of the same ligand or different ligands.

7. The method according to claim 1, wherein said at least one soluble ligand (ii) comprises multiple of the same ligand or different ligands.

8. The method according to claim 1, wherein one or more of said ligands or competing soluble analyte is labeled with a detectable label or marker.

9. The method according to claim 8, wherein said label is a fluorescent compound.

10. The method according to claim 1, wherein said biological sample contains cells of biological tissue.

11. The method according to claim 10, wherein said biological sample is selected from the group consisting of whole blood, urine, synovial fluid, bone marrow, cerebrospinal fluid, vaginal mucus, cervical mucus, sputum, semen, amniotic fluid, any cell-containing exudates, cell-containing media, and cell-containing buffer.

12. The method according to claim 10, further comprising diluting said sample with saline, buffer or a physiologically acceptable diluent prior to the additional of soluble ligand.

13. The method according to claim 1, wherein said cell type is selected from the group consisting of red blood cells, white blood cells, granulocytes, macrophages, platelets, lymphocytes, lymphoblasts, blast cells, leukocytes, neutrophils, fibroblasts, dendritic cells, epithelial cells, epidermal cells, embryonic cells, hepatocytes, histiocytes, peritoneal cells, kidney cells, lung cells, sperm cells, oocytes, normal and cancer cells of other mammalian tissue, and mixtures thereof.

14. The method according to claim 1, wherein said cellular target is selected from the group consisting of a cell surface antigen, an intercellular antigen, nuclear antigen, a fragment thereof, and mixtures thereof.

15. The method according to claim 1, wherein said soluble analyte is selected from the group consisting of a serum marker, a protein, a virus, a hormone, a lipid, a nucleic acid sequence, a carbohydrate, a pharmaceutical drug, a toxin, and an antigen shed from a cell.

16. The method according to claim 1, wherein said capture medium is a physiologically compatible bead.

17. The method according to claim 16, wherein said bead is from 0.05 to 20 microns in diameter.

18. The method according to claim 15, wherein said capture medium is greater than 1 μM in size.

19. The method according to claim 1, further comprising adding to said sample a reagent that inhibits phagocytosis of said capture medium without damaging said target cells or inhibiting binding to said target cells and said ligands.

20. The method according to claim 1, wherein each said ligand is independently selected from the group consisting of an antibody, an antigen, a nucleic acid sequence complementary to a target sequence, and an oligonucleotide sequence.

21. The method according to claim 1, further comprising washing said sample after combining said capture medium and said sample.

22. The method according to claim 1, wherein said sample contains blood cells, said method further comprising lysing said sample to remove red blood cells prior to said analyzing step.

23. The method according to claim 22, further comprising washing said sample after lysis.

24. The method according to claim 1, wherein said biological sample contains myeloid cells and wherein said method further comprises adding to said sample a reagent that inhibits phagocytosis of said capture medium by said myeloid cells.

25. The method according to claim 1, further comprising adding to said sample an inhibitor of cellular activation.

26. The method according to claim 25, further comprising adding the inhibitor of cellular activation with said sample prior to or simultaneously with the addition to the sample of said capture medium.

27. The method according to claim 26, wherein said inhibitor of cellular activation is selected from the group consisting of an anticoagulant, an inhibiting reagent, a fixative or an inhibiting reaction temperature below 25° C.

28. The method according to claim 1, wherein said method is useful in diagnosis of a disease or condition selected from the group consisting of sepsis, inflammation, autoimmune disease, cardiovascular disease, viral infection, bacterial infection, drug interaction.

29. The method according to claim 1, which is useful for evaluation of food or water for contamination with microorganisms or toxins.

30. A method for diagnosing sepsis or monitoring the progress thereof by performing the method of claim 1, wherein said cellular target is selected from the group consisting of CD64 (N), HLA-DR (Mo), CD11a, CD14/Cd16, CD142 (tissue factor); and said soluble target is selected from the group consisting of IL-6, IL-10, IL-1, TNF-alpha, neopterin, C-reactive protein, procalcitonin, Activated Protein C.

31. A method for diagnosing autoimmune disease or monitoring the progress thereof by performing the method of claim 1, wherein said cellular target is selected from the group consisting of activated T cells and activated B cells; and said soluble target is selected from the group consisting of C-reactive protein, a chemokine, and a cytokine.

32. A method for diagnosing cardiovascular disease or monitoring the progress thereof by performing the method of claim 1, wherein said cellular target is selected from the group consisting of platelet-leucocyte aggregates, CD142 (TF); and said soluble target is selected from the group consisting of hsC-reactive protein, troponin, and myoglobin.

33. A method for differential diagnosis of viral and bacterial infections or monitoring the progress thereof by performing the method of claim 1, wherein said cellular target is selected from the group consisting of HLA-DR, CD4/CD8, CD64(N), CD14/CD16; and said soluble target is selected from the group consisting of IFN-gamma, neopterin, and C-reactive protein.

34. A kit comprising:
(a) at least one soluble ligand that binds a cellular target in said sample;
(b) at least one soluble ligand that binds a soluble analyte in said sample or at least one soluble analyte associated with a detectable label; and
(c) a solid phase capture medium that binds directly to said soluble analyte, indirectly to said soluble analyte, or to said soluble ligand that binds to said soluble analyte.

35. The kit according to claim 34, further comprising a container comprising one or a mixture of components (a) through (c).

36. The kit according to claim 34, wherein said soluble ligand (a) is associated with a first detectable label; said solid phase capture medium (c) is associated with immobilized multiple first ligands that are capable of binding to said soluble analyte and wherein said capture medium-immobilized first ligand is capable of forming a first complex with said soluble analyte in a biological sample; and said soluble ligand (b) is associated with a second detectable label, and wherein said ligand (b) and said first complex are capable of forming a second complex.

37. The kit according to claim 34, wherein said ligand (a) is associated with a first label, wherein multiple of said ligand (a) are capable of binding to a single cellular target; said ligand (b) is associated with a second label; and wherein said solid phase capture medium (c) is associated with multiple of said soluble analytes.

38. The kit according to claim 34, wherein said ligand (a) is associated with a first label, wherein multiple of said ligand (a) are capable of binding to a single cellular target; said soluble analyte (b) is associated with a second label; and wherein said solid phase capture medium (c) is associated with multiple of said ligands capable of binding soluble analyte.

39. The kit according to claim 34, wherein said ligand (a) is associated with a first detectable marker; said at least one ligand (b) comprises a first ligand (b) associated with a second detectable marker and a second ligand (b); and said solid phase capture medium (c) comprises multiple immobilized ligands capable of binding to said first ligand (b) or second ligand (b).
